(19) United States
(12) Patent Application Publication
(10) Pub. No.: US 2025/0001137 A1
(43) Pub. Date: Jan. 2, 2025

(54) METHODS AND DEVICES FOR VASCULAR ACCESS

(71) Applicants: Thomas SUTTON, Summit, NJ (US); Joe RANALLETA, Greenville, SC (US); Nicholas Hogan MININNI, Wilmington, NC (US); Kevin JOHNSON, Hillsborough, NC (US)

(72) Inventors: Thomas SUTTON, Summit, NJ (US); Joe RANALLETA, Greenville, SC (US); Nicholas Hogan MININNI, Wilmington, NC (US); Kevin JOHNSON, Hillsborough, NC (US)

(21) Appl. No.: 18/344,568

(22) Filed: Jun. 29, 2023

(51) Int. Cl. A61M 25/00; A61M 25/06

(52) U.S. Cl. CPC A61M 25/0618; A61M 25/0075; A61M 25/0097; A61M 25/0606; A61M 25/0625; A61M 25/0631; A61M 2039/062; A61M 2039/064

(57) ABSTRACT

A catheter assembly configured for use with a medical device is provided herein. The catheter assembly can comprise a catheter hub having an interior chamber and a valve located in the interior chamber. The valve can comprise a distal portion, a proximal portion, and a sidewall extending therebetween to define a valve interior. The valve can include a barrier layer having a concave shape that extends in the valve interior from the sidewall at the distal portion. The barrier layer can have at least one slit extending therethrough that is in a normally closed configuration to prevent a fluid from passing through the barrier layer. The catheter assembly can further comprise a sealing ring located circumferentially at a distal end of the valve distally to the barrier layer. The sealing ring can form a seal between the valve and the interior chamber at the distal end.

### CROSS-REFERENCE TO RELATED APPLICATIONS

n/a

### FIELD OF THE INVENTION

The present disclosure is directed to valve assemblies having improved ability to selectively allow flow of fluids through the medical device and prevent leakage of fluids from the proximal end of the device. Such improved valve assemblies also prevent leakage of fluids after repeated insertion and removal of medical instruments through the valve, such as catheters, introducers, tubes, lines, and ports that can be used for vascular or other devices. The valve assemblies can also be used as a connection for needles (e.g., fistula needles), hemodialysis circuits, feeding tubes, urinary drain catheters, or any other suitable means.

### BACKGROUND

Catheters allow medical practitioners to administer infusion or removal of fluids from a patient. For example, catheters can function as a conduit that allows infusion of fluids, such as normal saline solution, therapeutic substances, and/or nutritional fluids into a patient. Alternatively, catheters can withdraw fluids such as blood or other bodily fluids as required by the particular medical procedure. In those cases where the medical practitioner intends to position the catheter into a vessel, the medical practitioner will look for a flow of blood back into the catheter (“flashback”) to verify placement of the catheter opening into a vessel. The number of different catheter insertion procedures and techniques is great and may include the use of a needle, dilator, stylet, or other medical devices within the catheter when placed.

Once properly positioned, the catheter's hub (or medical device positioned within the catheter) can be coupled to an adapter (typically a luer fitting) to enable fluid coupling of the catheter to the source of fluids or reservoir. However, in the case of an accidental disconnection between the catheter and the reservoir, there is a possibility for the patient to bleed out or to entrain air that will lead to an embolism, both of which are potentially life-threatening for the patient. Accidental disconnection can occur if the mating parts are not securely tightened. The mating parts can also become loose from patient movement, unwanted fidgeting, or other patient interference. Further, if the patients have any blood-borne pathogen (e.g., HIV, hepatitis, etc.), blood exposure for the caregiver is potentially lethal. As such, insertion of the catheter requires that the point of access remains sanitary. The period between insertion of the catheter and coupling of an adaptor can cause bodily fluids to escape through the catheter, causing an unsanitary condition for the medical practitioner who must handle the catheter for coupling of the adapter and/or remove the medical device inserted through the catheter. The caregiver often covers an open connection port with their finger to reduce the blood flow until making a mating connection. Since blood can be a medium for bacterial growth, infection chances can rise due to exposure at the time of catheter insertion.

As such, there remains a need for a valve assembly that permits controlled fluid flow that also reduces risk of infection by providing a tight seal with respect to the catheter. There also remains a need for a valve that slows blood loss to give the caretaker time to adequately clean the connection and wipe away any residual blood on the connection. There also remains a need for a valve that minimizes the blood exposure for the caregiver at the time of insertion, removal, or a change in catheters.

### SUMMARY

The illustrations and variations described herein are meant to provide examples of the methods and devices of the invention. It is contemplated that combinations of aspects of specific embodiments or combinations of the specific embodiments themselves are within the scope of this disclosure. The valve assemblies described herein can be used in any tubing assembly, especially medical tubing not only limited to catheter assemblies.

A catheter assembly configured for use with a male luer is provided. The catheter assembly can comprise a catheter hub having an interior chamber and a valve located in the interior chamber. The valve can comprise a distal portion, a proximal portion, and a sidewall extending therebetween to define a valve interior. The valve can include a barrier layer having an arcuate shape that extends in the valve interior from the sidewall at the distal portion. The barrier layer can have at least one slit extending therethrough that is in a normally closed configuration to prevent a fluid from passing through the barrier layer.

The catheter assembly can further comprise a sealing ring located circumferentially at a distal end of the valve distally to the barrier layer. The sealing ring can have an exterior sealing surface that forms a seal between the valve and the interior chamber at the distal end. An exterior diameter of the valve can be smallest at a recessed portion located between the sidewall and the barrier layer and proximally adjacent to the exterior sealing surface. The recessed portion can be configured to provide deformation of the valve. A thickness of the sidewall at the recessed portion can be less than a thickness of the barrier layer.

The catheter assembly can further comprise a protrusion on an exterior surface of the sidewall. The protrusion can be located proximally to the recessed portion. The protrusion can nest within an opening in the interior chamber such that upon insertion of the luer into the proximal portion of the valve, the protrusion reinforces an adjacent sidewall to reduce elastic deformation of the valve at the protrusion and causing increased outward elastic deformation of the sidewall at the recessed portion. Further distal advancement of the luer can cause elastic deformation of the barrier layer opening the at least one slit such that the fluid can pass through the barrier layer. A plurality of protrusions can be provided on the exterior surface of the sidewall. The plurality of protrusions can each couple with a plurality of openings within the interior chamber. A lubricant within the interior chamber can be further provided. As noted herein, the protrusions allow for any variation of the assemblies discussed herein to incorporate a valve within a hub without the need for any adhesives. Alternate variations can include the use of adhesives.

The sealing ring can comprise a rounded edge along a sealing ring circumference. This allows an option of assembling the devices herein without the use of additional fixtures to assemble the valve within a hub. The sealing ring can remain engaged with an inner surface of the chamber when the male luer engages the recessed portion. The valve can comprise a flange at a proximal end. The flange can engage with a proximal end of the catheter hub. A thickness of the sidewall can be less than the thickness of the barrier layer.

Another variation of the valves described herein includes a valve comprising a distal portion, a proximal portion, and a sidewall extending therebetween to define a valve interior, the valve including a barrier layer that extends in the valve interior from the sidewall at the distal portion, the barrier layer having a first contact surface, a second contact surface, and an interior recess therebetween, a slit extending through the barrier layer at the interior recess, wherein the slit is in a normally closed configuration that prevents a fluid from passing through the barrier layer, wherein upon insertion of the male luer into the valve interior, the male luer engages the first contact surface and the second contact surface to deform the barrier layer resulting in opening of the slit.

The present disclosure also includes a guard assembly for use with a tubing hub. In one variation the guard assembly includes a valve configured for positioning in the hub and comprising a distal portion, a proximal portion, and a sidewall extending therebetween to define a valve interior, the valve including a barrier layer at the distal portion having at least one slit extending therethrough that is in a normally closed configuration to prevent a fluid from passing through the barrier layer; a needle that extends through the hub and the slit of the barrier layer; a clip having a pair of arms extending from opposite ends of a rear wall of the clip, the rear arms being resilient, where each arm of the pair arms extends obliquely in a distal direction towards the needle such that a mid portion of each arm crosses the needle causing a distal segment of each arm to be biased against the needle on opposite sides of the needle, where a portion of each of the distal segment of each arm comprises an anchor section that engages the sidewall to anchor the clip to the valve interior during axial movement of the needle through an opening in the wall and between the distal segment of each arm; wherein withdrawal of a distal end of the needle proximally to the distal segment of each arm causes movement of the distal segment of each arm over the distal end of the needle to shield the distal end of the needle and also resulting in the anchor section of each arm to disengage from the sidewall; wherein continued proximal movement of the needle relative to the wall causes a stop surface on the needle to engage the opening in the wall such that the clip moves with the needle and remains with the needle when removed from the valve and the tubing hub.

The guard assembly can have an anchor assembly where the anchor section of each arm elastically deforms the sidewall, such that upon disengagement the sidewall returns to a pre-deformed condition.

Variations of the guard assembly can include an anchor section of each arm that comprises an edge of the distal segment opposite to an edge of the distal segment that is biased against the needle. In an additional variation, the anchor section of each arm comprises a protrusion on each distal segment that extends from a top edge opposite to an edge of the distal segment that is biased against the needle. In yet another variation, the anchor section of each arm comprises a protrusion on each distal segment that extends from each side of the distal segment.

Variations of the guard assembly can have a distal segment of each arm that is a curved lip.

In another variation, a guard assembly for use with a tubing hub includes a valve configured for positioning in the hub and comprising a distal portion, a proximal portion, and a sidewall extending therebetween to define a valve interior, the valve including a barrier layer at the distal portion having at least one slit extending therethrough that is in a normally closed configuration to prevent a fluid from passing through the barrier layer; a needle that extends through the hub and the slit of the barrier layer; and a clip having a first arms extending from an ends of a rear wall of the clip, the first arms being resilient, where the first arm extends obliquely in a distal direction towards the needle such that a mid portion of the first arm crosses the needle causing a distal segment the first arm to be biased against the needle, where a portion of the distal segment comprises an anchor section that engages the sidewall to anchor the clip to the valve interior during axial movement of the needle through an opening in the wall; wherein withdrawal of a distal end of the needle proximally to the distal segment causes movement of the distal segment over the distal end of the needle to shield the distal end of the needle and also resulting in the anchor section disengaging from the sidewall; wherein continued proximal movement of the needle relative to the wall causes a stop surface on the needle to engage the opening in the wall such that the clip moves with the needle and remains with the needle when removed from the valve and the tubing hub.

The present disclosure also includes a medical assembly for shielding a needle tip upon removal of a needle from a hub. For example, such a medical assembly includes a valve configured for positioning in an interior chamber of the hub and comprising a distal portion, a proximal portion, and a sidewall extending therebetween to define a valve interior, the valve including a barrier layer at the distal portion having at least one slit extending therethrough that is in a normally closed configuration to prevent a fluid from passing through the barrier layer; a clip having a rear wall with an opening that permits passage of the needle therethrough, the rear wall having a first end and a second end on radially opposing sides of the needle; a first arm extending obliquely and distally from the first end towards the needle, the first arm comprising a first arm section comprising a first dimension and a second arm section comprising a second dimension, wherein the first dimension is larger than the second dimension; a second arm extending obliquely and distally from the second end towards the needle, the second arm comprising a first arm section comprising a first dimension and a second arm section comprising a second dimension, wherein the first dimension is larger than the second dimension, where the first arm and the second arm are resilient; and where the first arm section of the first arm and the first arm section of the second arm each cross an axis of the needle such that the second arm section of the first arm and the second arm section of the second arm are on opposing sides of the needle and are biased against the needle; where an anchor portion of the second arm section of the first arm and an anchor portion of the second arm section of the second arm anchor into the sidewall of the valve by deforming the sidewall of the valve to prevent movement of the clip relative to the needle during axial movement of the needle through the rear opening of the clip.

In another variation, the present disclosure includes a needle catheter assembly. For example, the catheter assembly can include a catheter hub having an interior chamber; a valve located in the interior chamber and comprising a distal portion, a proximal portion, and a sidewall extending therebetween to define a valve interior, the valve including a barrier layer having at least one slit extending therethrough that is in a normally closed configuration to prevent a fluid from passing through the barrier layer; a needle positioned within the valve, the needle having a needle shaft terminating at a sharp tip at a distal needle end, the shaft further including a stop surface having an increased diameter relative to a regular diameter of the needle shaft; a clip having a rear wall at a proximal end, a first arm extends from a first end of the rear wall and a second arm extends from a second end of the rear wall, the first end and the second end being on opposite sides of the needle shaft; and the first arm having a first mid portion and the second arm having a second mid portion that both extend distally and laterally from the wall such that the first mid portion and second mid portion intersect a longitudinal needle axis of the needle shaft and extend away from the needle shaft; where a portion of a proximal end of the first arm extends laterally from the first mid portion towards the needle shaft to form a first needle guard and where a portion of a proximal end of the second arm extends laterally from the second mid portion towards the needle shaft to form a second needle guard, where the first arm and the second arm are resilient such that the first needle guard and second needle guard are biased against opposite sides of the needle shaft; wherein the first needle guard has a first anchor portion and the second needle guard has a second engagement surface, each of which engage the inner sidewall of the valve to prevent movement of the needle guard when the needle shaft moves through an opening in the rear wall and relative to the clip, wherein withdrawal of the distal end of the needle shaft proximally to the first needle guard and the second needle guard causes disengagement of the first engagement surface and the second engagement surface from the inner sidewall such that the first needle guard and second needle guard cover the sharp tip, wherein continued withdrawal of the needle shaft causes the stop surface to engage the rear wall such that the needle guard remains attached to the needle shaft upon removal of the needle shaft from the valve.

The present disclosure is related to the following commonly assigned patents and applications, the entirety of each of which is incorporated by reference: U.S. Pat. Nos.: U.S. Pat. No. 11,324,939 issued on May 10, 2022; U.S. Pat. Nos.: U.S. Pat. No. 8,105,288 issued on Jan. 31, 2012; U.S. Pat. No. 8,591,469 issued on Nov. 26, 2013; U.S. Pat. No. 9,775,973 issued on Oct. 3, 2017; U.S. Pat. No. 9,604,035 issued on Mar. 28, 2017; U.S. Pat. No. 10,828,465 issued on Nov. 10, 2020; U.S. publication nos.: U.S.20200016375A1 published on Jan. 16, 2020, and U.S.20210031009A1 published on Feb. 4, 2021; Provisional application nos.: 62/993,493 filed on Mar. 23, 2020; 63/037,496 filed on Jun. 10, 2020; and 63/037,841 filed on Jun. 11, 2020; U.S. Patent Application 63/367,403 filed Jun. 30, 2022; U.S. application Ser. No. 18/170,835 filed Feb. 17, 2023; U.S. application Ser. No. 17/812,397 filed Jul. 13, 2022, now U.S. Patent No. U.S. Pat. No. 11,607,525; U.S. application Ser. No. 17/822,975 filed Aug. 29, 2022; PCT international application no. PCT/US2023/069399 filed Jun. 29, 2023.

### BRIEF DESCRIPTION OF THE DRAWINGS

Each of the following figures diagrammatically illustrates aspects and variations to better understand the invention. Variation of the invention from the aspects shown in the figures is contemplated.

FIG. 1A shows a perspective view of a variation of an improved catheter-valve assembly.

FIG. 1B shows an exploded view of the assembly of FIG. 1A.

FIG. 2A shows a perspective view of one variation of a valve.

FIG. 2B shows a top view of the valve of FIG. 2A.

FIG. 2C shows a side view of the valve of FIG. 2A.

FIG. 2D shows a front view of the valve of FIG. 2A.

FIG. 2E shows a rear view of the valve of FIG. 2A.

FIGS. 3A and 3B show cross-sectional side views of a catheter hub and the valve of FIG. 1A.

FIGS. 3C and 3D show cross-sectional side views of a catheter hub and the valve of FIG. 1A with a male luer introduced therein.

FIG. 4A shows a perspective view of another variation of an improved catheter-valve assembly.

FIG. 4B shows an exploded view of the assembly of FIG. 4A.

FIG. 4C shows a perspective view of the valve of FIG. 4A.

FIG. 4D shows a side view of the valve of FIG. 4A.

FIG. 4E shows a cross-sectional side view of another variation of the catheter hub and the valve of FIG. 4A.

FIG. 4F shows a cross-sectional side view of another variation of the catheter hub and the valve of FIG. 4A with a male luer introduced therein.

FIGS. 4G and 4H show cross-sectional side views of a catheter hub and the valve of FIG. 4A with a male luer introduced therein.

FIGS. 5A to 5G show variations of valves having one or more contact surfaces on an interior surface of the barrier layer to assist in opening of the valve.

FIGS. 6A, 6B, and 6C illustrate a perspective view, top view, and side view respectively of another variation of a valve one or more elongated protrusions.

FIGS. 7A to 7C illustrate another variation of a valve having an elongated protrusion.

FIG. 8A shows another variation of a catheter-valve assembly having a catheter hub, a valve, and a needle inserted therethrough.

FIG. 8B shows a front perspective view and FIG. 8C shows a rear perspective view of the clip of FIG. 8A.

FIGS. 8D and 8E show partial cross-sectional view of a variation of the clip of FIGS. 8A to 8C positioned about a needle that extends through a valve, medical device hub, and back wall of the clip.

FIG. 8F shows a partial cross-sectional view the clip of FIG. 8E where the needle is withdrawn in a proximal direction relative to the hub and valve.

FIG. 8G illustrates the condition of the clip once the tip of the needle moves proximal to the needle guard sections.

FIGS. 9A and 9B show front and back perspective views of another variation of a clip.

FIG. 9C shows a reduced surface area of an anchor portion of the clip of FIGS. 9A and 9B that increase retention of the clip in the valve.

FIGS. 10A and 10B show front and back perspective views of another variation of a clip similar to the clips described above.

FIG. 10C shows another reduced surface area of an anchor portion of the clip of FIGS. 10A and 10B that increase retention of the clip in the valve.

FIGS. 11A and 11B illustrate partial cross-sectional views of a needle that extends within a hub, valve and a clip, where the clip is a single arm clip.

### DETAILED DESCRIPTION

For a better understanding of the present invention, reference will be made to the following description of the embodiments, which is to be read in association with the accompanying drawings, which are incorporated in and constitute a part of this specification, show certain aspects of the subject matter disclosed herein and, together with the description, help explain some of the principles associated with the disclosed implementations.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising. (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

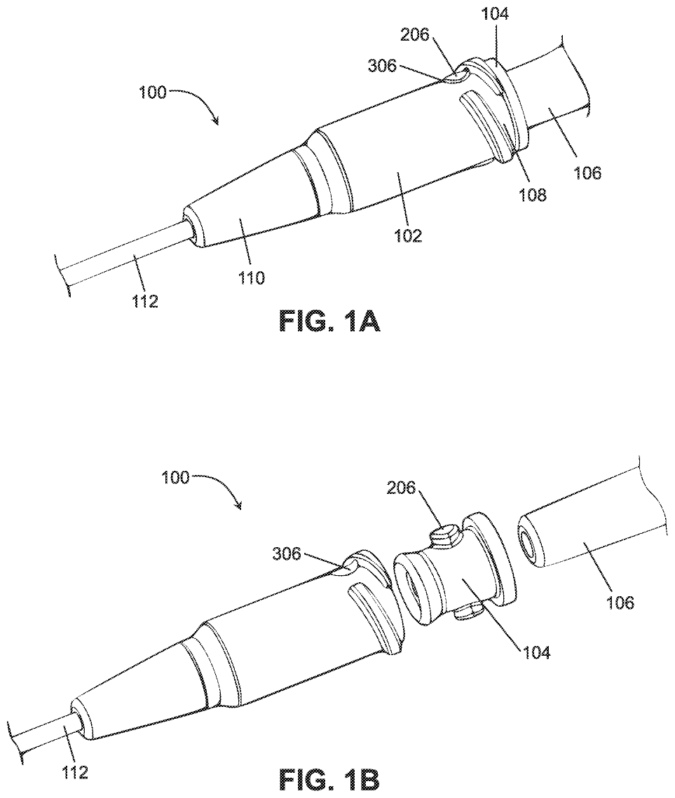

FIG. 1A shows catheter-valve assembly 100 having a catheter hub 102, a valve 104, and a male luer 106 inserted therethrough. The catheter hub 102 can comprise a proximal end 108 and a distal end 110. A catheter tubing 112 can be coupled to the catheter and can extend from the distal end 110. As seen in FIG. 1B and further described herein, the valve 104 is seated in the open proximal end 108 of the catheter hub 102 so that the male luer 106 can then be inserted through a proximal end of the valve 104. The valves described herein can be used with any medical device such as a needle or other component that allows fluid coupling. Such devices can include luers or hubs that are commercially used in the medical field. For purposes of this application, the male luer 106 can represent a medical device, such as a needle or other fluid source. As discussed below, the catheter hub 102 can include a pocket or hole 306 that nests protrusion 206 on the valve 104. The pocket 306 can extend partially within the catheter hub 102 or can extend through the catheter hub 102 forming a hole 306 as shown in FIGS. 1A and 1B.

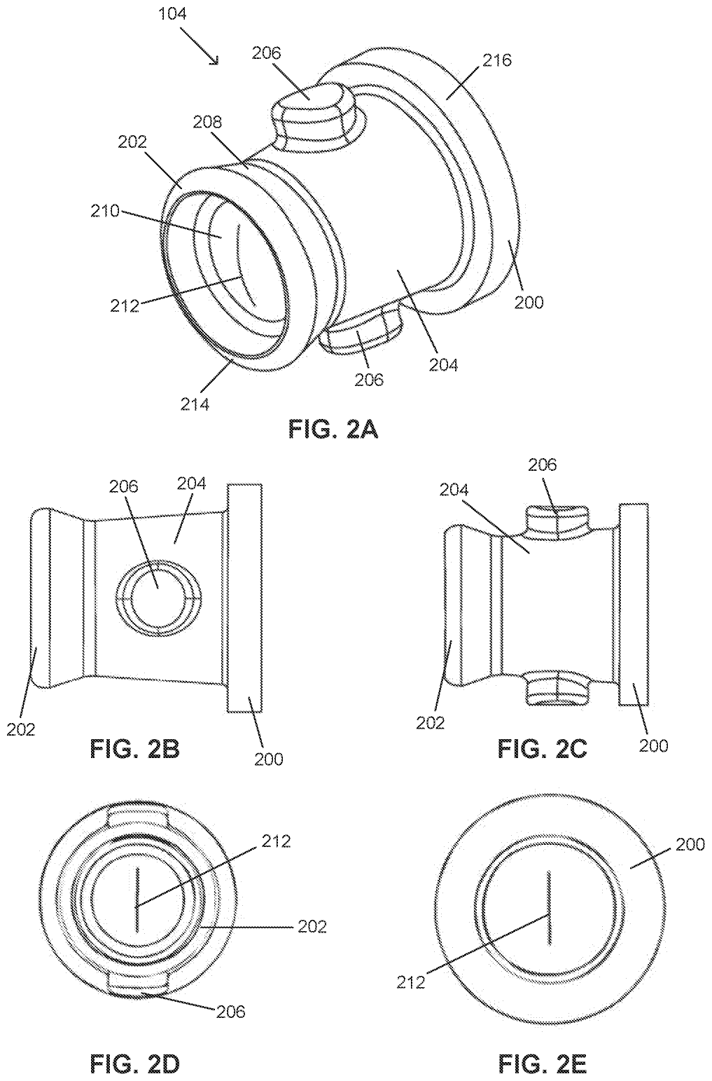

FIGS. 2A to 2E show a valve having proximal end 200, distal end 202, and a sidewall 204 therebetween. In this variation, the sidewall can have protrusions 206 positioned on the outer surface of the valve 104. Protrusions 206 can be on either side of the sidewall 204 and can be circular, though other shapes can be considered. Additionally, the valve 104 can have a recessed portion 208 distal to the protrusions 206 and the sidewall 204. The recessed portion 208 is located where the valve 104 has its smallest exterior diameter and allows the valve 104 to deform when engaged by the male luer 106. While the figure shows two protrusions 206, any number of protrusions are within the scope of this disclosure.

The valve 104 includes a barrier layer 210 that extends from the sidewalls in an interior of the valve to prevent fluid flow. Variations of the valve 104 comprise barrier layers 210 that are opened through elastic deformation rather than pressure within the catheter assembly. For example, variations of the valves 104 require the male luer 106 to elastically deform the valve 104 and/or barrier layer 210 to permit fluid flow through the valve 104. In one additional variation, the barrier layer includes an arcuate or concave shape in an interior of the valve. The barrier layer 210 includes at least one slit 212 extending therethrough and has a normally closed configuration to prevent a fluid from passing therethrough the barrier layer 210. Alternatively, the barrier layer 210 can have a plurality of slits 212 that form a plurality of leaflet structures or flaps that open upon deformation of the valve 104.

The barrier layer 210 generally includes a flexible or semi-flexible material that is compatible with exposure to blood, medicaments, and other fluids commonly encountered during catheterization/infusion procedures. The valve 104 can be fabricated from a compliable and resilient material such that insertion of the male luer 106 causes the barrier layer 210 to stretch and deform to an open configuration.

In an additional variation, a distal end 202 of a valve 104 can have a sealing ring 214 located circumferentially around the valve and distally to the barrier layer 210. As shown, variations of the valve can have a sealing ring 214 that includes a radius. This radius can allow for assembly of the valve 104 into a hub without additional fixtures. The sealing ring 214 can have an exterior sealing surface that forms a seal between the valve 104 and the hub 102 at the distal end 202 of the valve. The sealing ring 214 will be circumferentially engaged with the inner surface of the hub 102 such that the sealing ring 214 maintains a seal during insertion of the male luer 106, ensuring that fluid does not pass from the catheter hub 102 to an outer surface of valve 104. Variations of the valve can include a sealing ring 214 that is made of the same material as the remainder of the valve 104. Alternatively, the sealing ring 214 can comprise a different material than the remainder of the valve. Additionally, the angle at which the sealing ring 214 is offset with respect to the longitudinal axis can be changed to compress the valve 104 with respect to the hub 102 at varying diameters.

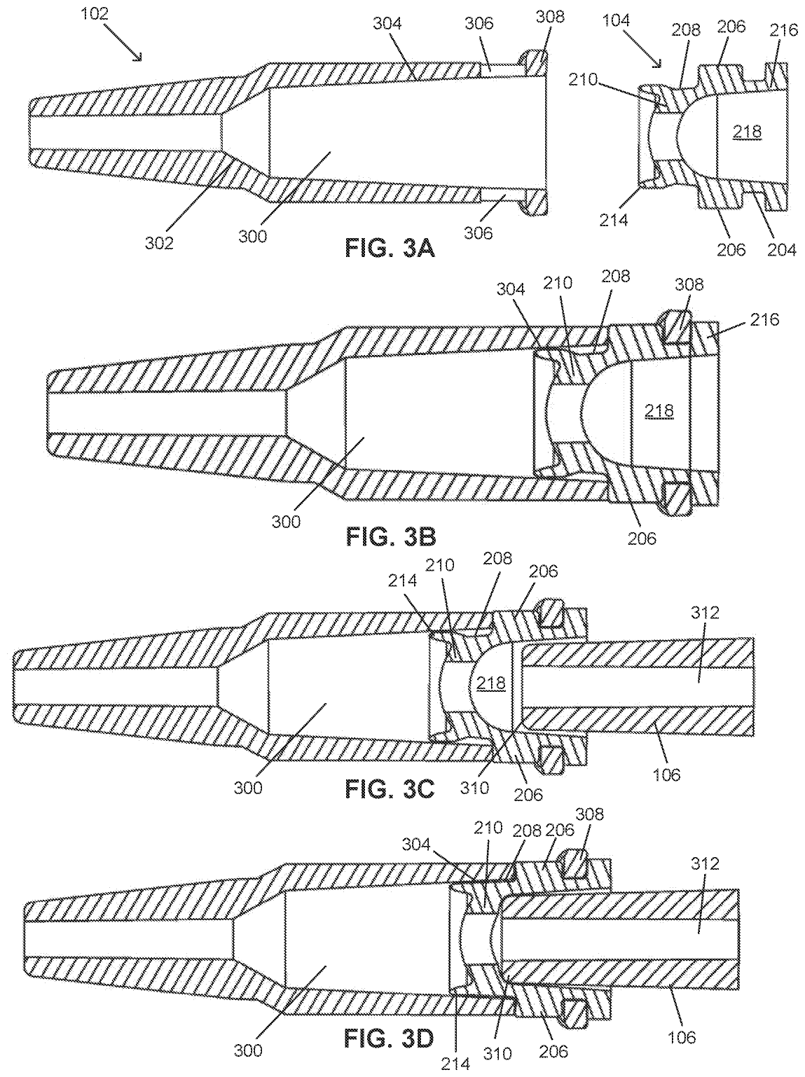

As seen in FIG. 3A, the catheter hub 102 has a chamber 300 extending from a proximal surface at the proximal end 108. The chamber 300 is in fluid communication with a catheter tubing 112 (FIGS. 1A and 1B) that is coupled to the hub 102. Variations of the chamber 300 can have straight walls or the walls can be tapered at an angle and can also have a tapered section 302 forming a female luer shape. The valve 104 exterior engages an inner surface 304 of the chamber 300 when the valve is inserted in the chamber, as seen in FIG. 3B. As shown in FIGS. 3B and 3C, the recessed portion 208 is offset from the inner surface 304 of the chamber 300. This offset in combination with the reinforcement of the valve wall adjacent to the protrusion 206 causes most of the deformation of the valve to occur at the recessed portion 208 of the wall distally to the protrusion.

Variations of the catheter hub 102 can comprise pockets or openings 306 positioned near the proximal end 108 of the hub 102, which seat the protrusions 206 of the valve 104 when the valve 104 is located in the chamber 300. Positioning of the valve 104 in the chamber 300 can occur via insertion or via molding of the valve into the chamber 300. As noted above, the protrusions 206 reinforce the sidewall 204 at the protrusions 206 to increase outward elastic deformation of the sidewall 204 at the recessed portion 208. The protrusions 206 nest in the openings 306 to hold the valve 104 in place such that the valve 104 to limit longitudinal stretching of the valve wall adjacent to the protrusion. The protrusions 206 in the present designs disclosed herein can also eliminate the need for any adhesive to hold the valve in place. This effect creates a preferential zone for stretching and/or deformation of the valve to open the barrier layer. The protrusions 206 in the present designs disclosed herein can also eliminate the need for any adhesive to hold the valve in place.

Protrusions 206 can additionally have a protrusion sealing ring that can provide additional interference along the openings 306. An outer portion of the protrusion where the protrusion 206 meets the edge of opening 306 accommodates the protrusion sealing ring. The protrusion sealing ring has a greater diameter than the remainder of the protrusion in order to provide a seal against the openings 306 of the hub 102. This seal, similar to sealing ring 214, prevents leaking of any fluid through the assembly.

Both the hub 102 and the valve 104 can comprise flanges, 216, 308 respectively, at their respective proximal ends that engage each other when the protrusions 206 nest within the openings 306. The valve flange portion 216 comprises a diameter greater than a diameter of the sidewall 204 for sealing against a proximal surface of the catheter hub flange 308. Alternatively, in some variations the valve flange portion 216 can include openings or segments such that it is not circumferentially continuous about the sidewall 204.

As seen in FIG. 3C, the male luer 106 is inserted into a valve interior 218 of the valve 104 and ultimately engages the walls/septum to elastically stretch the valve to open the barrier layer 210. The variations of the valve shown in FIG. 3C includes a valve interior 218 that is concave near the recessed portion 208 and includes walls with a tapered angle on the exterior and/or interior. During insertion, a distal end 310 of the male luer 106 can engage with the recessed portion 208 of the valve 104, as seen in FIG. 3D, causing the barrier layer 210 to open in a distal direction. Upon engagement and advancement of the luer, the barrier layer can undergo elastic deformation, opening the at least one slit 212 such that the fluid can pass through the barrier layer 210 and into a lumen 312 of the male luer 106. The valve interior 218 and male luer 106 can be provided with a small clearance therebetween to allow for easier longitudinal movement of the luer 106 within when the valve 104 diameter at the protrusions 206 decreases during insertion of the luer 106. In some variations, insertion of the male luer 106 into the valve interior 218 can cause the distal end of the barrier layer 210 to a fully open position, while the lateral surface of the male luer 106 engages the sidewall 204 of the valve 104. However, in alternate variations, the barrier layer 210 can merely deflect to allow sufficient fluid flow.

In additional variations of a valve, the thickness of the barrier layer 210 is greater than a thickness of the sidewall 204 at the recessed portion 208 as well as a thickness of the sidewall (excluding the regions where the protrusion 206 joins the sidewall). For example, the increased thickness of the barrier layer 210 permits the slit 212 to elastically return to a closed position once luer 106 is removed. The relatively thinner sidewall 204 reduces an offset distance between the internal diameter of the valve interior 218 and the inner surface 304 of chamber 300. Reducing this offset distance allows for insertion of the male luer 106 to a sufficient depth along a longitudinal axis to open the slit 212 at the barrier layer 210 of the valve 104 without being impeded by the sidewall 204 of the valve 104. For example, if the sidewall 204 is too thin, then the valve 104 can suffer from an increased risk of failure (e.g., cracking or splitting). The thickness of the barrier layer 210 increases relative rigidity in comparison with the remainder of the valve 104, allowing the slit 212 to close fully, increasing the likelihood that the slit returns to its original state to close the valve 104, preventing leakage. In some variations, the thickness differential also allows deformation of the valve to occur at the barrier layer rather than the sidewall or recessed portion 208.

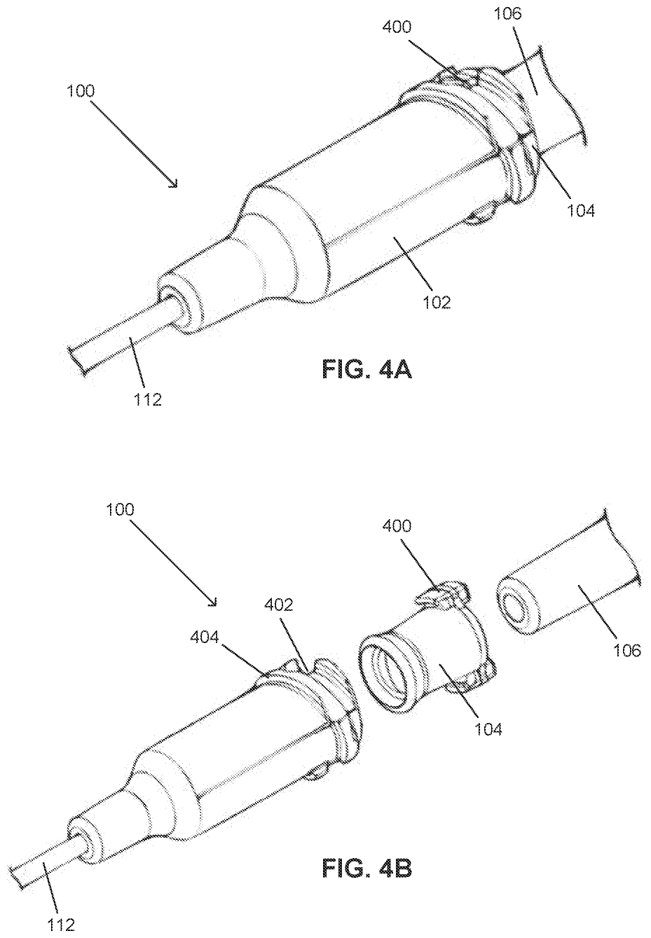

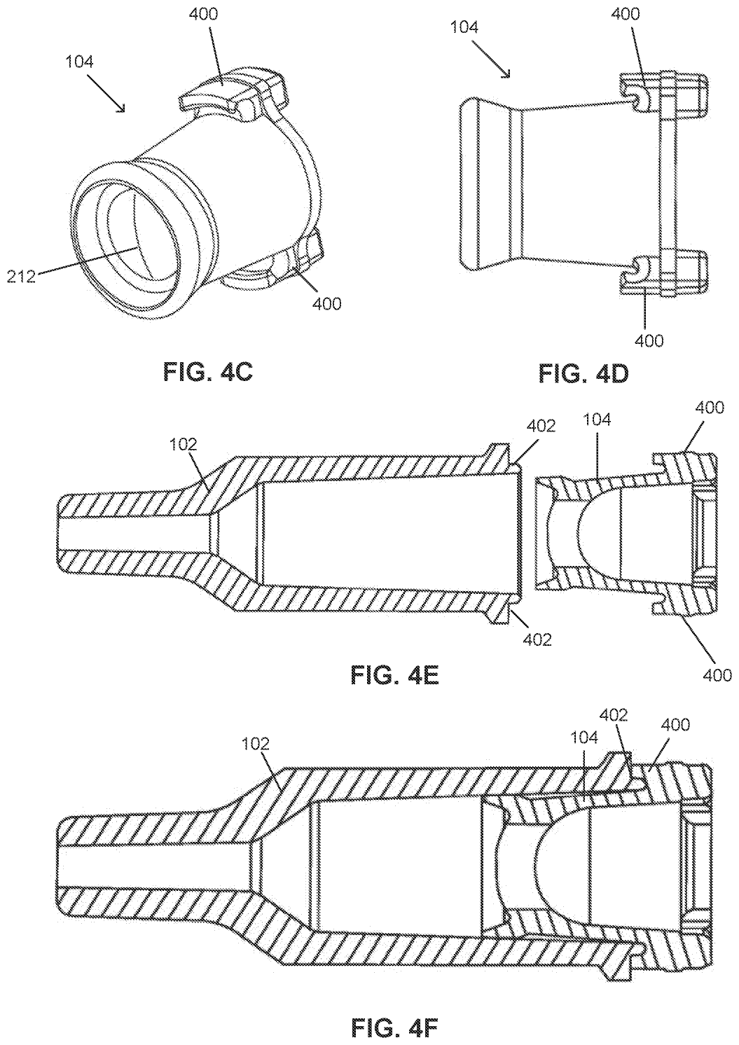

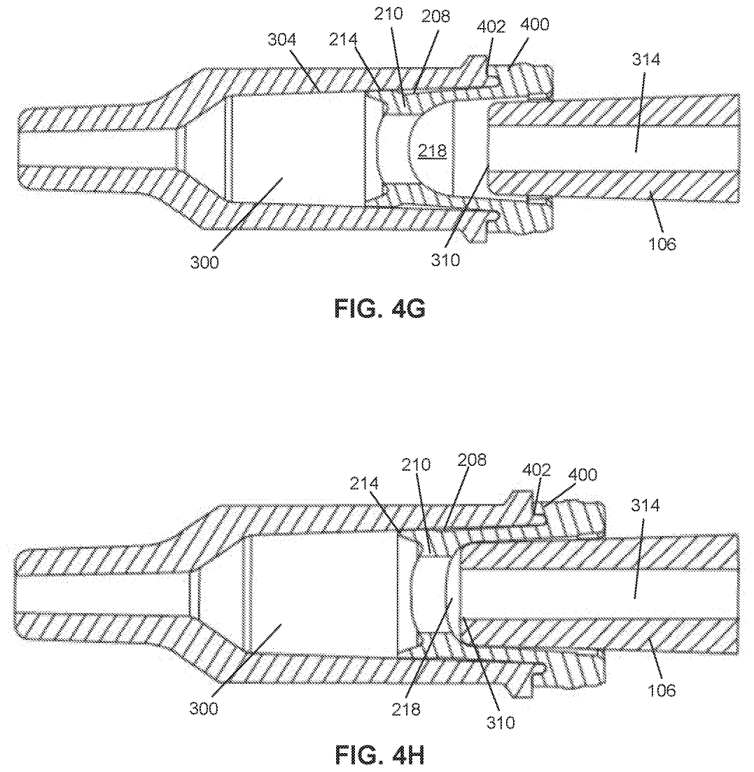

FIGS. 4A to 4H show various views of another variation of a catheter-valve assembly. In this variation, the valve 104 can have one or more lips 400 on a proximal end 200. The lips 400 can engage with one or more recesses 402 on the catheter hub 102, as seen in FIGS. 4G and 4H. Upon engagement with the recesses 402, the lips 400 can overhang onto the recesses 402 on the hub 102 to secure the valve 104 onto the hub 102 for use with the male luer 106 or another medical device. In another variation of the device, threads 404 on the catheter hub 102 can be used to hold lips 400, preventing the valve 104 from being pushed in too far into the catheter hub 102.

Similar to the features in the variation shown in FIGS. 3A to 3D, the catheter hub 102 can have a chamber 300 extending from a proximal surface at the proximal end 108. The chamber 300 is in fluid communication with a catheter tubing that is coupled to the hub 102. For purposes of illustration, the tubing is omitted from several of the figures. Variations of the chamber 300 can have straight walls or the walls can be tapered at an angle and can also have a tapered section 302 forming a female luer shape. The valve 104 exterior engages an inner surface 304 of the chamber 300 when the valve is inserted in the chamber, as seen in FIG. 4G. As shown in FIG. 4G, the recessed portion 208 is offset from the inner surface 304 of the chamber 300. This offset causes most of the deformation of the valve to occur at the recessed portion 208 of the wall.

As seen in FIGS. 4G and 4H, the male luer 106 is inserted into a valve interior 218 of the valve 104 and ultimately engages the walls/septum to elastically stretch the valve to open the barrier layer 210. The variations of the valve shown in FIGS. 4G and 4H includes a valve interior 218 that is concave near the recessed portion 208 and includes walls with a tapered angle on the exterior and/or interior. During insertion, a distal end 310 of the male luer 106 engages with the recessed portion 208 of the valve 104, as seen in FIG. 4H, causing the barrier layer 210 to open in a distal direction. Upon engagement and advancement of the luer, the barrier layer can undergo elastic deformation, opening the at least one slit 212 such that the fluid can pass through the barrier layer 210 and into a lumen 312 of the male luer 106. The valve interior 218 and male luer 106 can be provided with a small clearance therebetween to allow for easier longitudinal movement of the luer 106 within when the valve 104 diameter decreases during insertion of the luer 106. In some variations, insertion of the male luer 106 into the valve interior 218 can cause the distal end of the barrier layer 210 to a fully open position, while the lateral surface of the male luer 106 engages the sidewall 204 of the valve 104. However, in alternate variations, the barrier layer 210 can merely deflect to allow sufficient fluid flow.

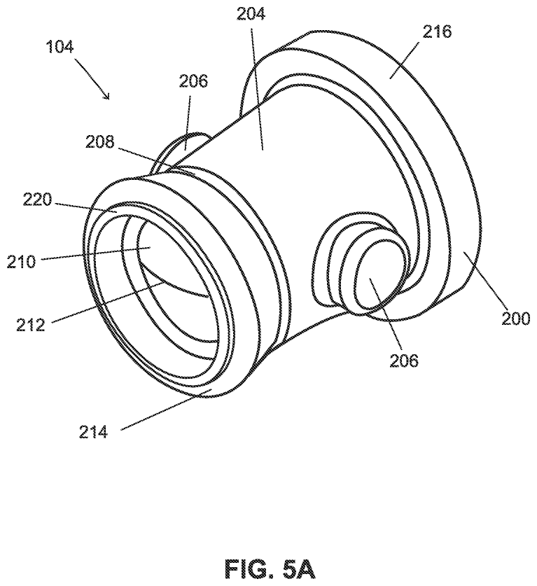

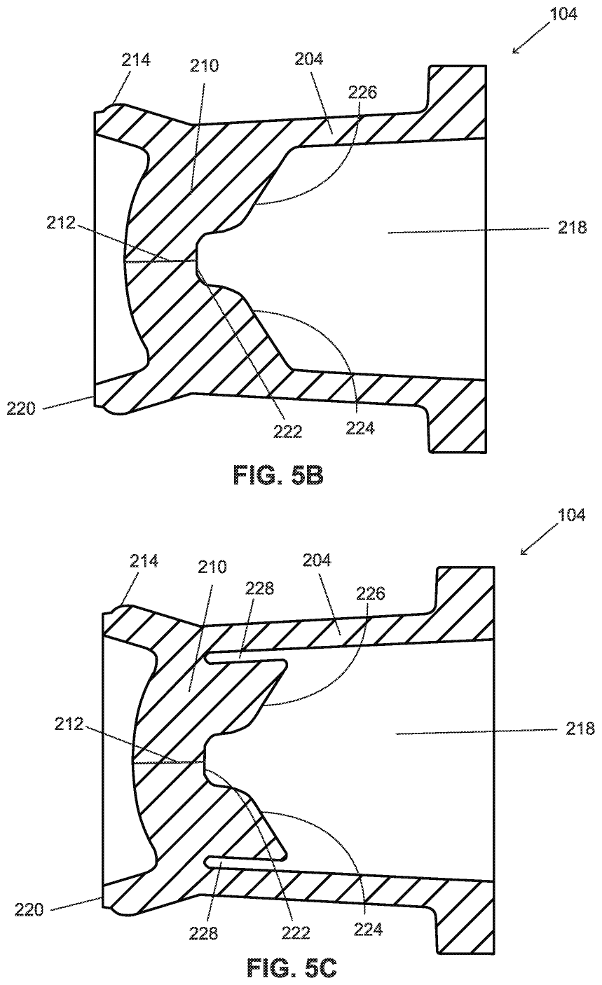

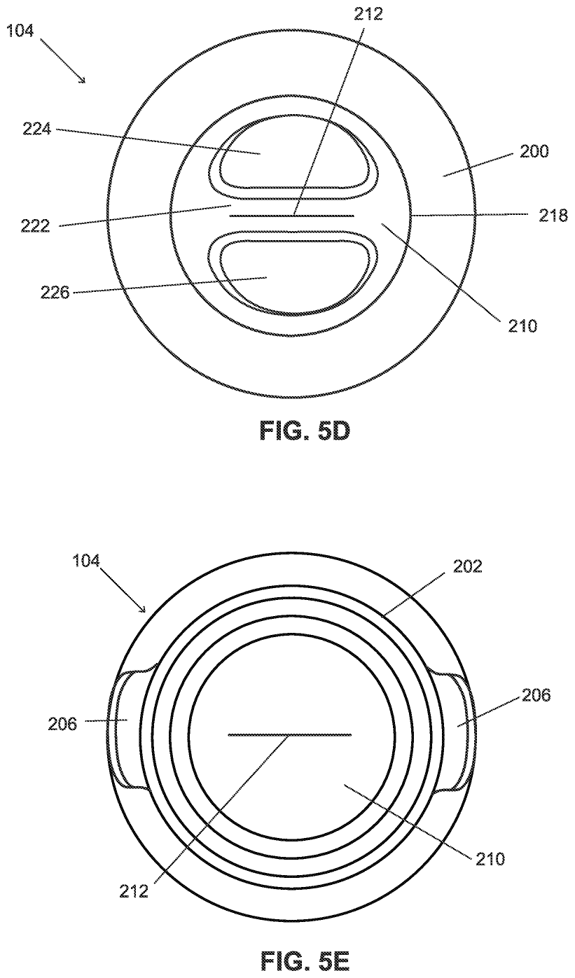

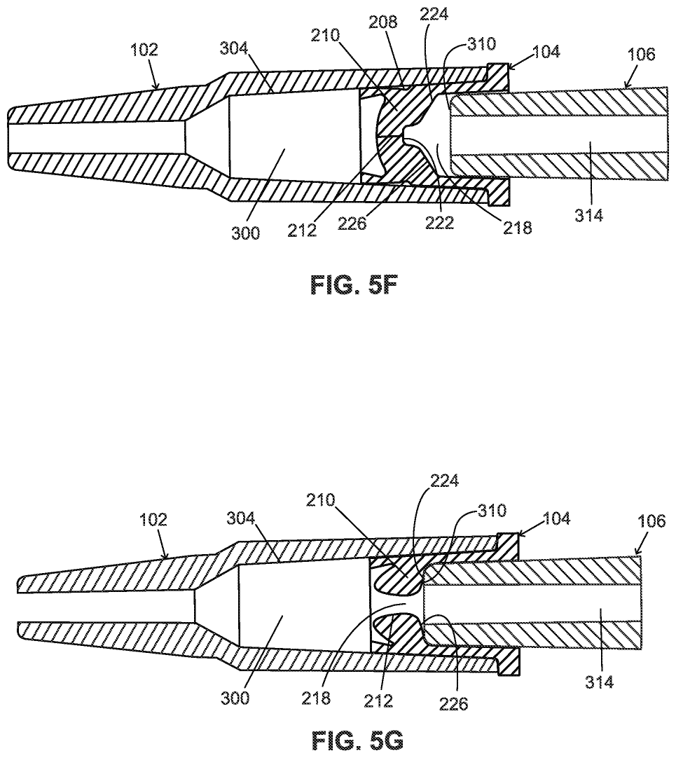

FIGS. 5A to 5G show a valve 104 having proximal end 200, distal end 202, and a sidewall 204 therebetween. As noted above, the sidewall 204 can have optional protrusions 206 positioned on the outer surface of the valve 104 on opposite sides of the sidewall 204 or valve 104. As noted above, the valve 104 can optionally include a recessed portion 208 distal to the protrusions 206 and the sidewall 204. The distal end 202 of the valve can include a scaling ring 214 with an optional flange 220.

As noted herein, the valves 104 include barrier layers 210 that are opened through clastic deformation rather than pressure within the valve 104 or catheter assembly (not shown). The variation of the valve shown in 5B, which is a cross-sectional view of the valve 104 of FIG. 5A includes a barrier layer 210 with an interior recess 222 for positioning of the slit 212. The presence of the interior recess 222 within the barrier layer 210 and adjacent to the valve interior 218 results in two contact surfaces 224, 226 or bumps on a surface of the barrier layer 210 that is in the valve interior 218. As shown, the barrier layer 210 can be solid such that any force applied on the contact surfaces 224, 226 causes deformation of the barrier layer 210, which deforms the slit 212 to open the valve. As illustrated, the barrier layer 210 can comprise a thicker dimension in the regions adjacent to the contact surfaces 224, 226 as compared to a thickness of the barrier layer 210 at the slit 212 or adjacent to the internal recess 222. In an additional variation as shown in FIG. 5C, the portion of the barrier layer 210 forming the contact surfaces 224, 226 can have a recess or space 228 separating a portion of the barrier 210 forming the contact surfaces 224, 228 from the wall 204.

In an additional variation, the slit 212 is aligned to extend in longitudinal alignment between the two protrusions 206. Since the internal recess 222 also runs parallel to the slit, the contact surfaces 224 and 226 deform portions of the valve 104 and sidewall 204 that are not supported by the protrusions 206. This results in selective deformation of the barrier layer 210 to open the slit 212. It is noted that variations of the valve 104 do not require the protrusions 206, the sealing ring 214, and/or the recessed portion 208. Such variations can include the internal recess 222 alone or in combination with any of the previously mentioned features.

FIG. 5D illustrates a rear view showing a valve interior 218 of the valve 104 of FIGS. 5A and 5B showing the proximal end 200 of the valve 104. As shown, the slit 212 is positioned in the internal recess 222 located within the barrier layer 210. The formation of the internal recess 222 results in a first and second contact surfaces 224 226 that are on either side of the slit 212 and internal recess 222. FIG. 5E illustrates a front view showing a distal end 202 of the valve 104 of FIG. 5A. As illustrated, the slit 212 is formed through the barrier layer 210 and extends in alignment between the two protrusions 206.

FIGS. 5F and 5G illustrate cross sectional views of a valve 104 as shown in FIGS. 5A and 5B inserted into a medical device hub 102 where a male luer 106 is inserted into the interior 218 of the valve 104. As discussed above, the valve 104 includes a barrier layer 210 having an internal recess 222 located adjacent to an interior 218 of the valve 104. A slit 212 is positioned in the interior recess 222 and extends through the barrier layer 210. The barrier layer 210 also includes first and second contact surfaces 224 226. As shown, the valve 104 is in a normally closed configuration such that the barrier layer 210 provides a fluid seal or block between the chamber 300 of the hub 102 and the fluid path 314 of the male luer 106. FIG. 5F illustrates the condition prior to the distal end 310 of the male luer engaging the barrier layer 210 of the valve 104. As noted, the valve 104 can optionally include a recessed portion 208 that assists in deformation of the barrier layer 210. However, the outer wall of the valve 104 engages the inner surface 304 of the hub 102.

FIG. 5G shows the condition where the distal end 310 of the male luer 106 advances against contact surfaces 224 and 226 of the barrier layer 210 causing deformation of the barrier layer 210 to open the slit 212 causing the chamber 300 of the hub 102 to be fluidly coupled to the fluid path 314 of the male luer 106. Engagement of the luer 106 against the barrier layer 210 causes elastic deformation of the valve side wall and barrier layer 210 of the valve 104, which elastically deforms the slit 212 to open the valve 104.

In additional variations of a valve, the thickness of the barrier layer 210 is greater than a thickness of the sidewall 204 at the recessed portion 208 as well as a thickness of the sidewall. For example, the increased thickness of the barrier layer 210 permits the slit 212 to elastically return to a closed position once luer 106 is removed. The relatively thinner sidewall 204 reduces an offset distance between the internal diameter of the valve interior 218 and the inner surface 304 of chamber 300. Reducing this offset distance allows for insertion of the male luer 106 to a sufficient depth along a longitudinal axis to open the slit 212 at the barrier layer 210 of the valve 104 without being impeded by the sidewall 204 of the valve 104. For example, if the sidewall 204 is too thin, then the valve 104 can suffer from an increased risk of failure (e.g., cracking or splitting). The thickness of the barrier layer 210 can increase relative rigidity in comparison with the remainder of the valve 104, allowing the slit 212 to close fully, increasing the likelihood that the slit returns to its original state to close the valve 104, preventing leakage. In some variations, the thickness differential also allows deformation of the valve to occur at the barrier layer rather than the sidewall or recessed portion 208.

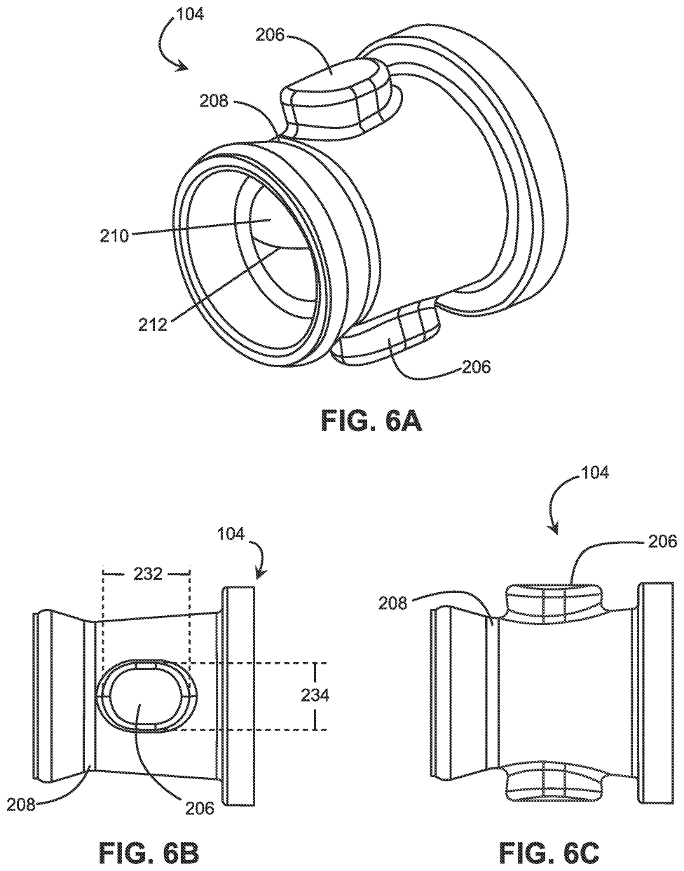

FIGS. 6A, 6B, and 6C illustrate a perspective view, top view, and side view respectively of another variation of a valve 104 of the present disclosure having one or more elongated protrusions 206 positioned on the outer surface of the valve 104. As noted above, the protrusions 206 can be positioned symmetrically on the side of the sidewall 204, or they can be asymmetrically positioned, and can be positioned proximal to a recessed portion 208 as shown in FIG. 6A to 6C. As noted above, any number of protrusions 204, are within the scope of this disclosure. In the variation shown in FIGS. 6B, the valve 104 includes at least one protrusion 206 having an elongated shape where a length along an axis of the valve (e.g., an axial length 232) is greater than a width along a circumference of the valve (i.e., a circumferential width 234). This elongated protrusion 206 supports a longer length of the internal wall of the valve to prevent deformation as discussed above. In addition, as shown in FIG. 6C, the protrusion 206 is located proximally to the minimum diameter of the recessed portion 208. The valve 104 also shows a slit 212 in a barrier layer 210 as extending transversely to the protrusions 206. As noted herein, variations include a valve slit 212 that extends in alignment between the protrusions 206. The recessed portion 208 has a minimum diameter and allows the valve 104 to deform when engaged by a medical device inserted into the interior chamber while the protrusions 206 limit deformation of the wall portion that is adjacent to the protrusion.

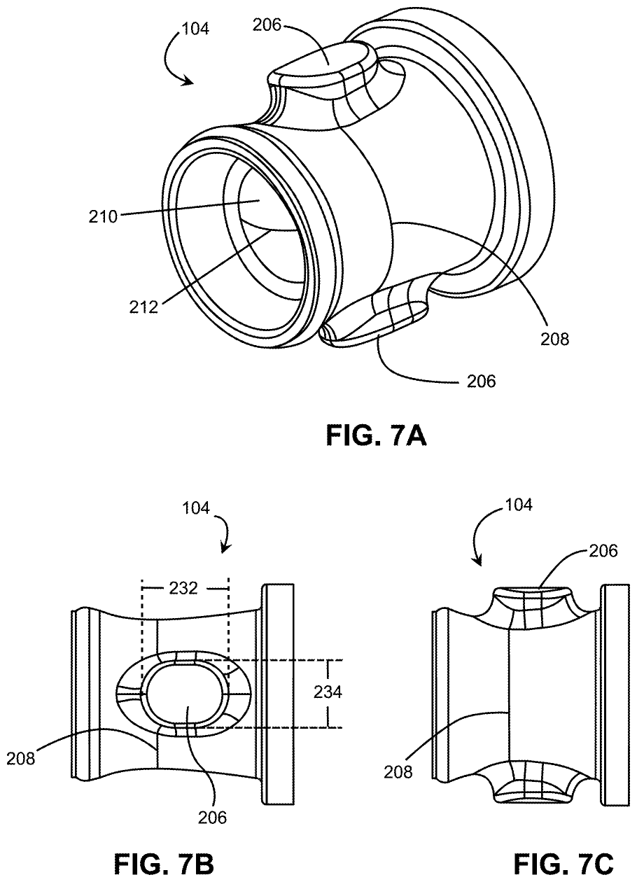

FIGS. 7A to 7C illustrate another variation of a valve 104 having an elongated protrusion 206 where the axial length 232 is greater than a width 234 in the circumferential direction 23. In this variation, the protrusion illustrates a valve similar to the valve shown in FIGS. 6A to 6C where the valve 104 includes elongated protrusions 206 that have a length along an axis or axial length 232 that is greater than a width along a circumference or a circumferential width 234. In the variation shown in FIGS. 7A to 7C, one or more of the protrusions 206 extends over the minimum diameter of the recessed portion 208.

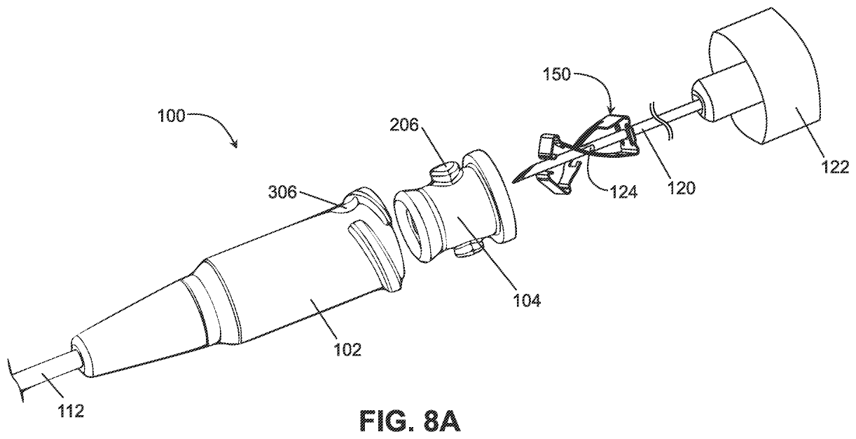

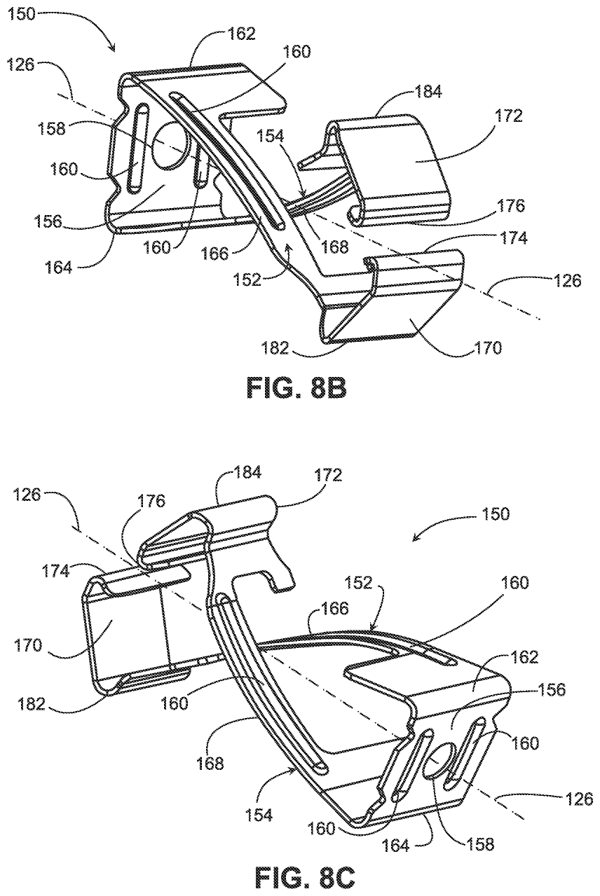

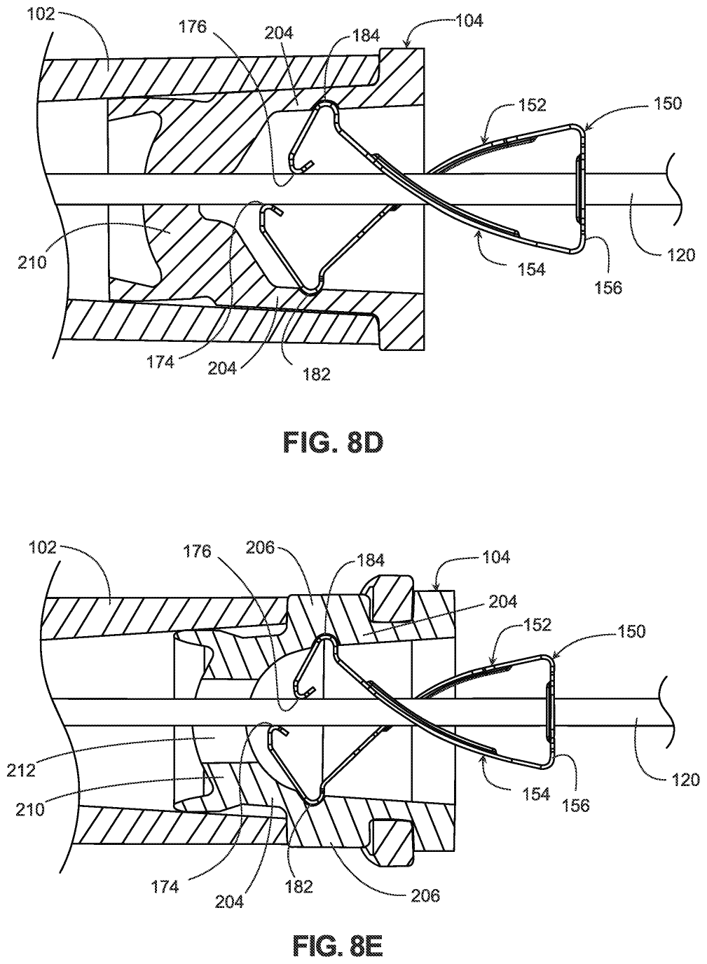

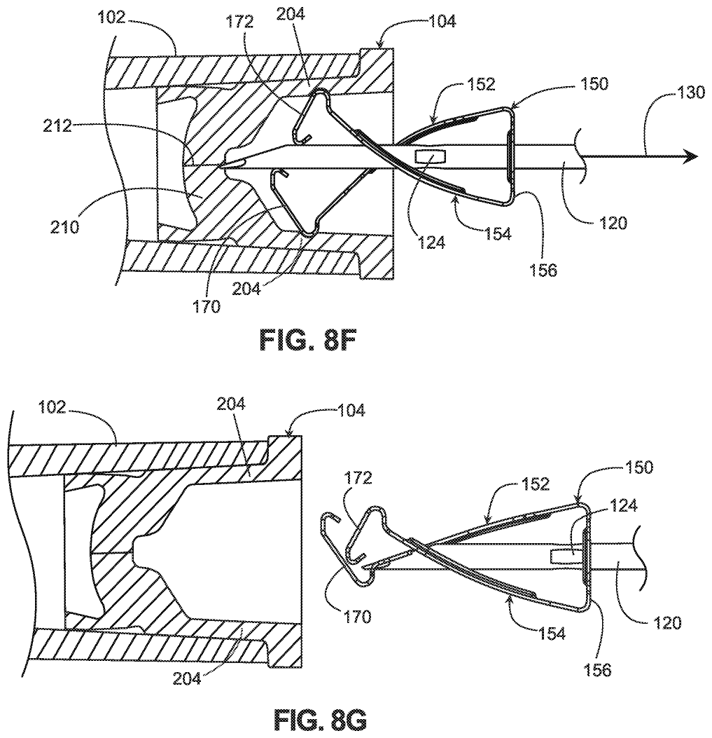

FIG. 8A shows another variation of a catheter-valve assembly 100 having a catheter hub 102, a valve 104, and a needle 120 inserted therethrough. Variations of the needle 120 can include a standard hub 122 that couples to threads on the catheter hub 102. The needle 120 can include a clip 150 that functions as a needle guard as described below. In addition, the needle can include a stop surface 124, which can have a raised diameter relative to a remainder of the needle 120 or can comprise a material affixed to the needle 120.

FIG. 8B shows a front perspective view and FIG. 8C shows a rear perspective view of the clip 150 of FIG. 8A. As shown, this variation of the clip 150 includes a first arm 152 and a second arm 154 each attached or continuous with a rear wall 156. The clip 152 can be a unitary construction or can have components that are attached to form the clip. As shown in FIGS. 8B and 7C, the first arm 152 extends from a first end 162 of the rear wall 156 and the second arm 154 extends from a second end 164 of the rear wall 156. Typically, when mounted on a needle (not shown in FIGS. 8B or 8C) the needle extends through the opening 158 such that the first 162 and second ends 164 on opposite sides of the needle. For purposes of illustrating features of the clip 150 the needle is omitted. In its place, a longitudinal needle axis 126 is shown to represent the needle. In addition, directionally from the back wall 156 towards the front of the clip 150 is a proximal-to-distal direction of the needle and clip 150.

As shown, the arms 152, 154 extend distally along the needle axis 126 and the mid portions 166 168 also extend in an oblique or lateral angle from the ends 162, 164 of the wall 156 towards the needle axis 126 such that the mid portions 166 168 intersect the longitudinal needle axis 126 and continue to extend away longitudinal needle axis 126 until a distal segment/end portion 170 of the first arm 152 and a distal segment/end portion 172 of the second arm 154 engage the needle on opposite sides from their location of the rear wall 156.

The distal segments/end portions 170 172 of the arms 152, 154 continue to extend obliquely or laterally their respective mid portions 166, 168 back towards the longitudinal needle axis 126 such that the respective needle guards that have ends 174 176 that bias against the needle due to the resiliency of the arms 152 152 and/or clip 150. In the clip 150 variation shown in FIGS. 8B and 8C, the distal portion or ends 174 176 of one or both of the distal segments 170 172 comprises a curved lip configuration that can further assist in covering a tip of a needle. Moreover, a width of each needle guards can be wider than a diameter of the needle. In an additional variation, the arms 152 154 comprise regions of varying widths across a length of the arms 152 154.

The clip 150 described herein also include one or more anchor portions that engage a valve wall to prevent movement of the clip 150 during movement of the needle. In the variation of FIGS. 8B and 8C, an edge of the first needle guard 170 and an edge 184 of the second needle guard 172 serve as the anchor portions. In additional variations, the anchor portions can be roughened or otherwise treated to increase a grip against the valve wall.

In the variation shown in FIGS. 8B and 8C, the clip 150 can include any number of reinforcement structures 160 that cause preferential bending of the clip at desired locations. For example, a reinforcement structure 160 can comprise a stiffening member positioned on the clip 150 or can comprise a rib having an increased thickness that is manufactured or deposited on a surface of the clip 150.

FIGS. 8D and 8E show partial cross-sectional view of a variation of the clip 150 of FIGS. 8A to 8C positioned about a needle 120 that extends through a valve 104, medical device hub 102, and back wall 156 of the clip 150. As shown, the needle 120 can extend through the barrier layer 210 and slit 212. Accordingly, the needle 120 will move relative to the hub 102, valve 104 and clip 150. As shown, the ends of the needle guards 174 176 on the first 152 and second 154 arms are biased against the needle 120 where the anchor portions, in this case the edges 182 184 of the needle guards engage the valve wall 204 to anchor the clip 150 within the valve 104. The deformation of the valve wall 204 shown in the figures is intended for illustrative purposes only. Variations of the devices can include clips 150 that deform a valve wall 204 to varying degrees. FIG. 8D illustrates the anchor portions 182 184 that are engaged with a portion of the valve wall 204 that does not contain a protrusion. Alternatively, FIG. 8E illustrates the anchor portions 182 184 engaging the valve wall 204 at an area adjacent to the protrusions 206.

FIG. 8F shows a partial cross-sectional view the clip 150 of FIG. 8E where the needle 120 is withdrawn in a proximal direction 130 relative to the hub 102 and valve 104. The arms 152 154 of the clip 150 remain engaged with the wall 204 of the valve 104 to prevent movement of the clip 150 with the needle. Accordingly, the needle 120 slides through the opening of the rear wall 156 and through the needle guard sections 170 172 of the clip 150. As shown, the stop surface 124 of the needle 120 has yet to engage the clip 150.

FIG. 8G illustrates the condition of the clip 150 once the tip of the needle moves proximal to the needle guard sections 170 172. Since the arms 152 154 are resilient (i.e., they elastically deform), withdrawal of the needle 120 causes the needle guards 170 172 to move together to effectively shield the tip of the needle 120. Since the arms 150 154 pivot close together, they disengage from the wall 204 of the valve 104 allowing the wall to return to its normal configuration. As shown, needle guard section 170 overlaps needle guard section 172 distally in front of the needle 120 tip forming a barrier that prevents inadvertent contact with the needle tip and also prevents distal movement of the needle tip. Continued movement of the needle 120 within the clip 150 causes the stop surface 124 to engage the rear wall 156 such that the clip 150 remains attached to the needle 120 upon removal of the needle from the valve and hub 102.

Use of a valve 104 to anchor the clip 150 effectively secures the clip 150 within the valve 104 due to the ability of the valve wall 204 to deform. This prevents inadvertent release of the clip as compared to a groove or other seat in a relatively stiff material such as a catheter hub.

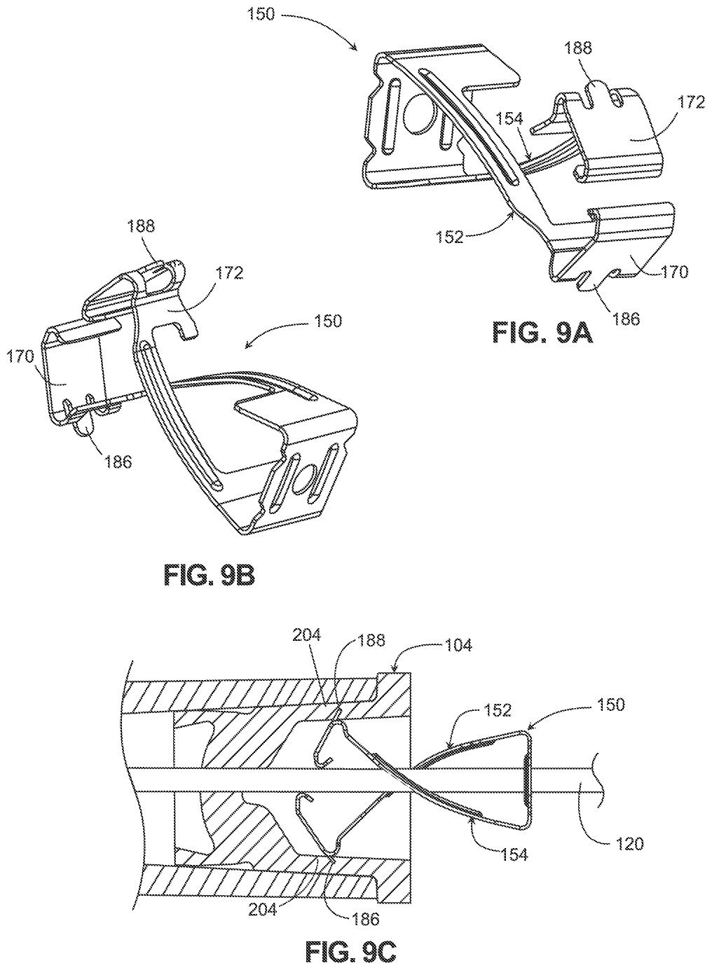

FIGS. 9A and 9B show front and back perspective views of another variation of a clip 150 similar to the clips 150 described above. In this variation, the clip 150 includes a first and second arms 152 154 with needle guards 170 172 having anchor portions that comprise fingers 186 188 that extend outwards such that they engage a wall of the valve. These anchor portions 186 188 provide reduced surface areas relative to the edges 182 184 of the clip 150 of FIGS. 8A to 8C. Accordingly the reduced surface area allows the anchor portions to apply more stress to the wall 204 of the valve 104 to increase retention of the clip 150 as shown in FIG. 9C.

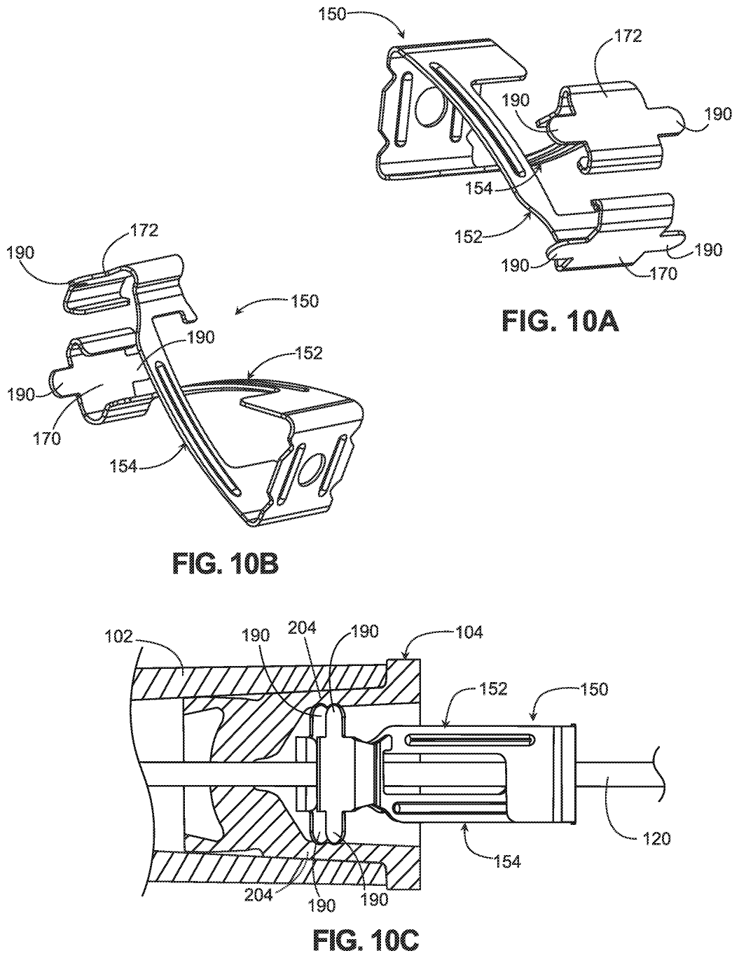

FIGS. 10A and 10B show front and back perspective views of another variation of a clip 150 similar to the clips 150 described above. In this variation, the clip 150 includes a first and second arms 152 154 with needle guards 170 172 having one or more wings 190 that extend from a side of the needle guards 170 172 and function as anchor portions. As with the variation of FIG. 9A, the side wings 190 have a reduced surface area that increase a stress on the valve 104 wall to anchor the clip 150 within the valve 104, shown in the top-view of the clip 150 in FIG. 10C.

It is intended that any of the anchor portions described herein can be combined onto a single spring. Moreover, the shapes of the anchor portions illustrated above are for exemplary purposes only, the anchor portions can take a variety of shapes as required.

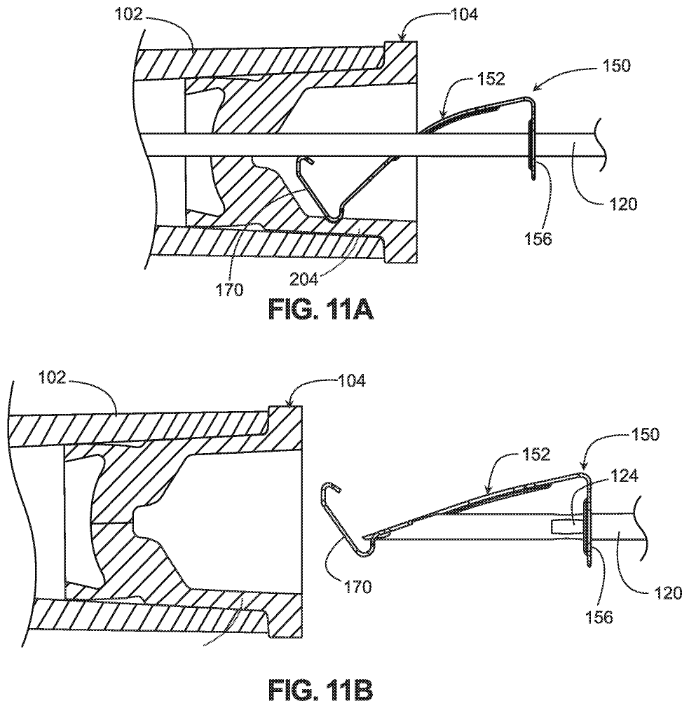

FIGS. 11A and 11B illustrate partial cross-sectional views of a needle 120 that extends within a hub 102, valve 104 and a clip 150, where the clip is a single arm 152 clip 150. The clip 150 of FIGS. 11A and 11B functions similarly to the clips 150 described above, as the rear wall 156 accepts the needle 120 and the single arm 152 extends from an end of the wall 156. The single arm 152 includes a single needle guard 170 hat engages a wall 204 of the valve 104. FIG. 11B illustrates a configuration where the needle 120 is withdrawn from the hub 102 and valve 104, and the single needle guard 170 moves over the needle 120 tip when the needle is moved proximally to the guard 170. As noted above, further withdrawal of the needle 120 causes the stop surface 124 to engage the rear wall 156 so that the clip 150 remains with the needle 120. While FIGS. 11A and 11B do not show any reduce surface anchors, such features can be combined with the single arm clip 150.

It is contemplated that any variation or aspect of a variation can be combined with any other variation (e.g., the disclosure includes combinations of the various features of the valves, clips, hubs, etc. shown above even if such features are shown separately in the illustrations).

The previous description of the disclosed embodiments is provided to enable any person skilled in the art to make or use the present invention. Various modifications to these embodiments will be readily apparent to those skilled in the art, and the generic principles defined herein can be applied to other embodiments without departing from the spirit or scope of the invention. For example, a wide variety of materials may be chosen for the various components of the embodiments. It is therefore desired that the present embodiments be considered in all respects as illustrative and not restrictive, reference being made to the appended claims as well as the foregoing descriptions to indicate the scope of the invention.

1. A guard assembly for use with a tubing hub, the guard assembly comprising:
a valve configured for positioning in the tubing hub and comprising a distal portion, a proximal portion, and a sidewall extending therebetween to define a valve interior, the valve including a barrier layer at the distal portion having at least one slit extending therethrough that is in a normally closed configuration to prevent a fluid from passing through the barrier layer;
a needle that extends through the tubing hub and the at least one slit of the barrier layer;
a clip having a pair of arms extending from opposite ends of a rear wall of the clip, the pair of arms being resilient, where each arm of the pair of arms extends obliquely in a distal direction towards the needle such that a mid portion of each arm crosses the needle causing a distal segment of each arm to be biased against the needle on opposite sides of the needle, where a portion of each of the distal segment of each arm comprises an anchor section that engages the sidewall to anchor the clip to the valve interior during axial movement of the needle through an opening in the rear wall and between the distal segment of each arm;
wherein withdrawal of a distal end of the needle proximally to the distal segment of each arm causes movement of the distal segment of each arm over the distal end of the needle to shield the distal end of the needle and also resulting in the anchor section of each arm to disengage from the sidewall;
wherein continued proximal movement of the needle relative to the rear wall causes a stop surface on the needle to engage the opening in the rear wall such that the clip moves with the needle and remains with the needle when removed from the valve and the tubing hub.

2. The guard assembly of claim 1, wherein the anchor section of each arm elastically deforms the sidewall, such that upon disengagement the sidewall returns to a pre-deformed condition.

3. The guard assembly of claim 1, wherein the anchor section of each arm comprises an edge of the distal segment opposite to an edge of the distal segment that is biased against the needle.

4. The guard assembly of claim 1, wherein the anchor section of each arm comprises a protrusion on each distal segment that extends from a top edge opposite to an edge of the distal segment that is biased against the needle.

5. The guard assembly of claim 1, wherein the anchor section of each arm comprises a protrusion on each distal segment that extends from each side of the distal segment.

6. The guard assembly of claim 1, wherein the distal segment of each arm that is biased against the needle comprises a curved lip.

7. The guard assembly of claim 1, wherein a width of the distal segment of each arm is greater than a diameter of the needle.

8. The guard assembly of claim 1, where the distal segment of each of the arms overlap.

9. The guard assembly of claim 1, where the distal segment of each of the arms are equidistant from the rear wall.

10. The guard assembly of claim 1, wherein the valve comprises a first contact surface, a second contact surface, and an interior recess therebetween, the at least one slit extending through the barrier layer at the interior recess, wherein the at least one slit is in a normally closed configuration that prevents the fluid from passing through the barrier layer, wherein upon application of a force on the first contact surface and the second contact surface deforms the barrier layer resulting in opening of the at least one slit.

11. The guard assembly of claim 1, wherein the valve comprises a sealing ring located circumferentially at a distal end of the valve distally to the barrier layer, the sealing ring having an exterior sealing surface that forms a seal between the valve and an interior chamber of the tubing hub at the distal end of the valve, wherein an exterior diameter of the valve is smallest at a recessed portion located between the sidewall and the barrier layer and proximally adjacent to the exterior sealing surface, the recessed portion being configured to provide deformation of the valve, and where a thickness of the sidewall at the recessed portion is less than a thickness of the barrier layer.

12. The guard assembly of claim 1, wherein the valve comprises a protrusion on an exterior surface of the sidewall, where the protrusion nests within an opening in the tubing hub such that upon insertion of a medical device into the proximal portion of the valve, the protrusion reinforces an adjacent sidewall to reduce elastic deformation of the valve at the protrusion and causing increased outward elastic deformation of the sidewall at a recessed portion, wherein further distal advancement of the medical device causes elastic deformation of the barrier layer opening the at least one slit such that the fluid can pass through the barrier layer.

13. A medical assembly for shielding a needle tip upon removal of a needle from a hub, the medical assembly comprising:
a valve configured for positioning in an interior chamber of the hub and comprising a distal portion, a proximal portion, and a sidewall extending therebetween to define a valve interior, the valve including a barrier layer at the distal portion having at least one slit extending therethrough that is in a normally closed configuration to prevent a fluid from passing through the barrier layer;
a clip having a rear wall with an opening that permits passage of the needle therethrough, the rear wall having a first end and a second end on radially opposing sides of the needle;
a first arm of the clip extending obliquely and distally from the first end towards the needle, the first arm comprising a first arm section comprising a first dimension and a second arm section comprising a second dimension, wherein the first dimension is larger than the second dimension;
a second arm of the clip extending obliquely and distally from the second end towards the needle, the second arm comprising a first arm section comprising a first dimension and a second arm section comprising a second dimension, wherein the first dimension is larger than the second dimension, where the first arm and the second arm are resilient; and
where the first arm section of the first arm and the first arm section of the second arm each cross an axis of the needle such that the second arm section of the first arm and the second arm section of the second arm are on opposing sides of the needle and are biased against the needle;
where an anchor portion of the second arm section of the first arm and an anchor portion of the second arm section of the second arm engage the sidewall of the valve by deforming the sidewall of the valve to prevent movement of the clip relative to the needle during axial movement of the needle through the opening in the rear wall.

14. The medical assembly of claim 13, wherein the anchor portion of the first arm and the anchor portion of the second arm both elastically deforms the sidewall, such that upon disengagement the sidewall returns to a pre-deformed condition.

15. The medical assembly of claim 13, wherein the anchor portion of the first arm and the anchor portion of the second arm comprises an edge opposite to an edge that is biased against the needle.

16. The medical assembly of claim 13, wherein the anchor portion of the first arm or the anchor portion of the second arm comprises a protrusion that extends from a top edge opposite to an edge of the second arm section that is biased against the needle.

17. The medical assembly of claim 13, wherein the anchor portion of the first arm or the anchor portion of the second arm comprises one or more wings that extends from each side of the clip.

18. The medical assembly of claim 13, wherein a distal portion of at least the first arm or the second arm that is biased against the needle comprises a curved lip.

19. The medical assembly of claim 13, further comprising one or more reinforcement structures on the clip.

20. The medical assembly of claim 13, wherein a width of the second arm section of the first arm and a width of the second arm section of the second arm is greater than a diameter of the needle.

21. The medical assembly of claim 13, wherein the second arm section of the first arm and the second arm section of the second arm overlap.

22. The medical assembly of claim 13, wherein the second arm section of the first arm and the second arm section of the second arm are equidistant from the rear wall.

23. A needle catheter assembly, the needle catheter assembly comprising:
a catheter hub having an interior chamber;
a valve located in the interior chamber and comprising a distal portion, a proximal portion, and a sidewall extending therebetween to define a valve interior, the valve including a barrier layer having at least one slit extending therethrough that is in a normally closed configuration to prevent a fluid from passing through the barrier layer;
a needle positioned within the valve, the needle having a needle shaft terminating at a sharp tip at a distal needle end, the needle shaft further including a stop surface having an increased diameter relative to a regular diameter of the needle shaft;
a clip having a rear wall at a proximal end, a first arm extends from a first end of the rear wall and a second arm extends from a second end of the rear wall, the first end and the second end being on opposite sides of the needle shaft; and
the first arm having a first mid portion and the second arm having a second mid portion that both extend distally and laterally from the rear wall such that the first mid portion and second mid portion intersect a longitudinal needle axis of the needle shaft and extend away from the needle shaft;
where a portion of a distal end of the first arm extends laterally from the first mid portion towards the needle shaft to form a first needle guard and where a portion of a distal end of the second arm extends laterally from the second mid portion towards the needle shaft to form a second needle guard, where the first arm and the second arm are resilient such that the first needle guard and second needle guard are biased against opposite sides of the needle shaft;
wherein the first needle guard has a first anchor portion and the second needle guard has a second anchor portion, each of which engage the sidewall of the valve to prevent movement of the first needle guard and second needle guard when the needle shaft moves through an opening in the rear wall and relative to the clip, wherein withdrawal of a distal end of the needle shaft proximally to the first needle guard and the second needle guard causes disengagement of the first anchor portion and the second anchor portion from the sidewall such that the first needle guard and the second needle guard cover the sharp tip, wherein continued withdrawal of the needle shaft causes the stop surface to engage the rear wall such that the clip remains attached to the needle shaft upon removal of the needle shaft from the valve.

24. The needle catheter assembly of claim 23, wherein the first anchor portion and the second anchor portion both elastically deforms the sidewall, such that upon disengagement the sidewall returns to a pre-deformed condition.

25. The needle catheter assembly of claim 23, wherein the first anchor portion and the second anchor portion comprises an edge opposite to an edge that is biased against the needle.

26. The needle catheter assembly of claim 23, wherein the first anchor portion or the second anchor portion comprises a protrusion that extends from a top edge opposite to an edge that is biased against the needle.

27. The needle catheter assembly of claim 23, wherein the first anchor portion or the second anchor portion comprises one or more wings that extends from each side of the clip.

28. The needle catheter assembly of claim 23, wherein a distal portion of at least the first needle guard or the second needle guard that is biased against the needle comprises a curved lip.

29. The needle catheter assembly of claim 23, further comprising one or more reinforcement structures on the clip.

30. The needle catheter assembly of claim 23, wherein a width of the first needle guard and a width of the second needle guard is greater than a diameter of the needle.
